(19) United States
(12) Patent Application Publication
(10) Pub. No.: US 2022/0331310 A1
(43) Pub. Date: Oct. 20, 2022

(54) METHODS FOR ALLEVIATING PTERYGIUM-ASSOCIATED WORRY ABOUT EYE APPEARANCE

(71) Applicant: Cloudbreak Therapeutics, LLC, Irvine, CA (US)

(72) Inventors: Jinsong Ni, Irvine, CA (US); Scott Whitcup, Irvine, CA (US); Rong Yang, Mission Viejo, CA (US)

(21) Appl. No.: 17/640,889

(22) PCT Filed: Sep. 10, 2020
(86) PCT No.: PCT/US2020/050150

§ 371 Date: Mar. 7, 2022

Related U.S. Application Data
(60) Provisional application No. 62/898,401, filed on Sep. 10, 2019.

(51) Int. Cl. A61K 31/496; A61K 9/00; A61P 27/02

(52) U.S. Cl. CPC A61K 31/496; A61K 9/0048; A61P 27/02

(57) ABSTRACT

Methods for reducing anxiety such as worrying or bothering about the ocular disease and/or eye appearance in pterygia patients are described. The methods can include administration of a multikinase inhibitor, for example nintedanib, to patients in need thereof.

### CROSS-REFERENCE TO RELATED APPLICATIONS

This application claims priority to U.S. Provisional Application Ser. No. 62/898,401, filed on Sep. 10, 2019, incorporated herein by reference in its entirety.

### TECHNICAL FIELD

Provided herein are materials and methods for the alleviation of worry about an ocular disease and/or eye appearance, for example, worry about the ocular disease and/or eye appearance because of changes in the eye associated with an ocular disease such as pterygium.

### BACKGROUND

Pterygium is a non-malignant fibrovascular growth that originates from the nasal or temporal conjunctiva, then advances onto the corneal surface. Pterygium often causes ocular and psychological symptoms that significantly impact patients' quality of life. Currently, there is no approved therapeutic drug product to treat pterygium. Surgical excision is used to remove pterygia lesion due to vision impairment and/or unsightly eye appearance.

### SUMMARY

Provided herein are materials and methods for the alleviation of worry about eye appearance, for example, worry about eye appearance because of an ocular disease such as pterygium.

In one aspect, provided herein is a method of reducing worry or anxiety about appearance of an affected eye in a subject, wherein the affected eye is affected with pterygium, hyperemia in pterygium, hyperemia, pinguecula, or pseudopterygium, the method including identifying a subject with worry or anxiety about eye appearance and administering a therapeutically effective amount of a multikinase inhibitor to the affected eye of the subject for a period of time.

Implementations can include one or more of the following features. A rating of worry or anxiety about eye appearance, as determined by a patient questionnaire, by the subject can decrease between (a) before administering a therapeutically effective amount of a multikinase inhibitor to an affected eye of the subject to a period of time and (b) after administering a therapeutically effective amount of a multikinase inhibitor to an affected eye of the subject to a period of time. The rating can be on a numerical scale. The rating can decrease by at least about 25%. The rating can decrease by at least about 30%. The rating can decrease by at least about 35%. The rating can decrease by at least about 40%. The rating can decrease by at least about 45%. The rating can decrease by at least about 50%. The rating can decrease by at least about 55%. The rating can decrease by at least about 60%. The rating can decrease by at least about 65%. The rating can decrease by at least about 70%. The rating can decrease by at least about 75%. The numerical scale can be a five-point scale. The rating can decrease by at least about 0.5. The rating can decrease by at least about 0.7. The rating can decrease by at least about 0.9. The rating can decrease by at least about 1.0. The rating can decrease by at least about 1.1. The rating can decrease by at least about 1.3. The rating of worry or anxiety about eye appearance, as determined by a patient questionnaire, can be based on a question regarding whether the worry about the appearance of the affected eye has impacted the quality of life of the subject in the last week. The rating of worry or anxiety about eye appearance, as determined by a patient questionnaire, can be solely based on a question regarding whether the worry about the appearance of the affected eye has impacted the quality of life of the subject in the last week. The rating of worry or anxiety about eye appearance, as determined by a patient questionnaire, can include the category, “Has the appearance of the affected eye impacted the quality of life during the last week?”, and the question “Worry about eye appearance?”. The rating of worry or anxiety about eye appearance, as determined by a patient questionnaire, can be solely based on the category, “Has the appearance of the affected eye impacted the quality of life during the last week?”, and the question “Worry about eye appearance?”. The questionnaire can be derived, at least in part, from the Visual Functioning Questionnaire-25 (VFQ-25) questionnaire. The questionnaire can be derived, at least in part, from the Ocular Surface Disease Index (OSDI) questionnaire. The multikinase inhibitor can be selected from afatinib, amuvatinib, axitinib, cabozantinib, canertinib, cediranib, ceritinib, crenolanib, crizotinib, dabrafenib, dacomitinib, dasatinib, erlotinib, foretinib, gefitinib, golvatinib, ibrutinib, icotinib, idelalisib, imatinib, lapatinib, lenvatinib, neratinib, nilotinib, nintedanib, palbociclib, pazopanib, ponatinib, quizartinib, regorafenib, ruxolitinib, sorafenib, sunitinib, tandutinib, tivantinib, tivozanib, trametinib, vandetanib, vatalanib, vemurafenib, or combinations thereof. The multikinase inhibitor can be selected from axitinib, nintedanib, regorafenib, and pazopanib. The multikinase inhibitor can be axitinib. The multikinase inhibitor can be nintedanib. The multikinase inhibitor can be pazopanib. The multikinase inhibitor can be a free base. The multikinase inhibitor can be a pharmaceutically acceptable salt.

In another aspect, provided herein is a method of reducing one or more patient reported signs or symptoms in a patient having an eye affected with pterygium, hyperemia in pterygium, hyperemia, pinguecula, or pseudopterygium, including administering a therapeutically effective amount of a multikinase inhibitor to the affected eye of the patient.

Implementations can include one or more of the following features. The patient reported sign or symptom can be worry or anxiety about eye appearance in the patient. Reducing the one or more patient reported signs or symptoms can include a measured reduction in the one or more patient reported signs or symptoms of at least about 10%, at least about 20%, at least about 30%, at least about 40%, at least about 50%, at least about 60%, or at least about 70% as compared to a control, a non-treated patient, or a baseline of the patient prior to administration of the multikinase inhibitor. The therapeutically effective amount of a multikinase inhibitor can be administered to the affected eye of the subject for a period of time. Reducing the one or more patient reported signs or symptoms can include a measured reduction in the one or more patient reported signs or symptoms as compared to a control. Reducing the one or more patient reported signs or symptoms can include a measured reduction in the one or more patient reported signs or symptoms as compared to a non-treated patient. Reducing the one or more patient reported signs or symptoms can include a measured reduction in the one or more patient reported signs or symptoms as compared to a baseline of the patient prior to administration of the multikinase inhibitor. Reducing the one or more patient reported signs or symptoms can include a measured reduction in the one or more patient reported signs or symptoms of at least about 25%. Reducing the one or more patient reported signs or symptoms can include a measured reduction in the one or more patient reported signs or symptoms of at least about 30%. Reducing the one or more patient reported signs or symptoms can include a measured reduction in the one or more patient reported signs or symptoms of at least about 35%. Reducing the one or more patient reported signs or symptoms can include a measured reduction in the one or more patient reported signs or symptoms of at least about 40%. Reducing the one or more patient reported signs or symptoms can include a measured reduction in the one or more patient reported signs or symptoms of at least about 45%. Reducing the one or more patient reported signs or symptoms can include a measured reduction in the one or more patient reported signs or symptoms of at least about 50%. Reducing the one or more patient reported signs or symptoms can include a measured reduction in the one or more patient reported signs or symptoms of at least about 55%. Reducing the one or more patient reported signs or symptoms can include a measured reduction in the one or more patient reported signs or symptoms of at least about 60%. Reducing the one or more patient reported signs or symptoms can include a measured reduction in the one or more patient reported signs or symptoms of at least about 65%. Reducing the one or more patient reported signs or symptoms can include a measured reduction in the one or more patient reported signs or symptoms of at least about 70%. Reducing the one or more patient reported signs or symptoms can include a measured reduction in the one or more patient reported signs or symptoms of at least about 75%. Reducing the one or more patient reported signs or symptoms can include a measured reduction in the one or more patient reported signs or symptoms on a five-point numerical scale. Reducing the one or more patient reported signs or symptoms can include a measured reduction of least 0.5 in the one or more patient reported signs or symptoms on a numerical scale. Reducing the one or more patient reported signs or symptoms can include a measured reduction of at least 0.7 in the one or more patient reported signs or symptoms on a numerical scale. Reducing the one or more patient reported signs or symptoms can include a measured reduction of at least 0.9 in the one or more patient reported signs or symptoms on a numerical scale. Reducing the one or more patient reported signs or symptoms can include a measured reduction of at least 1.0 in the one or more patient reported signs or symptoms on a numerical scale. Reducing the one or more patient reported signs or symptoms can include a measured reduction of at least 1.0 in the one or more patient reported signs or symptoms on a numerical scale. Reducing the one or more patient reported signs or symptoms can include a measured reduction of at least 1.3 in the one or more patient reported signs or symptoms on a numerical scale. The patient reported sign or symptom can determined by a patient questionnaire that includes a question regarding whether worry about the appearance of the affected eye has impacted the quality of life of the subject in the last week. The patient reported sign or symptom can be determined by a patient questionnaire and can be solely based on a question regarding whether worry about the appearance of the affected eye has impacted the quality of life of the subject in the last week. The patient reported sign or symptom can be determined by a patient questionnaire that includes the category, “Has the appearance of the affected eye impacted the quality of life during the last week?”, and the question “Worry about eye appearance?”. The patient reported sign or symptom can be determined by a patient questionnaire and is solely based on the category, “Has the appearance of the affected eye impacted the quality of life during the last week?”, and the question “Worry about eye appearance?”. The patient reported sign or symptom can be determined by a patient questionnaire that is derived, at least in part, from the Visual Functioning Questionnaire-25 (VFQ-25) questionnaire. The patient reported sign or symptom can be determined by a patient questionnaire that is derived, at least in part, from the Ocular Surface Disease Index (OSDI) questionnaire. The multikinase inhibitor can be selected from afatinib, amuvatinib, axitinib, cabozantinib, canertinib, cediranib, ceritinib, crenolanib, crizotinib, dabrafenib, dacomitinib, dasatinib, erlotinib, foretinib, gefitinib, golvatinib, ibrutinib, icotinib, idelalisib, imatinib, lapatinib, lenvatinib, neratinib, nilotinib, nintedanib, palbociclib, pazopanib, ponatinib, quizartinib, regorafenib, ruxolitinib, sorafenib, sunitinib, tandutinib, tivantinib, tivozanib, trametinib, vandetanib, vatalanib, vemurafenib, or combinations thereof. The multikinase inhibitor is selected from axitinib, nintedanib, regorafenib, and pazopanib. The multikinase inhibitor can be axitinib. The multikinase inhibitor can be nintedanib. The multikinase inhibitor can be pazopanib. The multikinase inhibitor can be a free base. The multikinase inhibitor can be a pharmaceutically acceptable salt.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a plot of the comparison of change of score on a questionnaire of worry about eye appearance to subjects between nintedanib and placebo via topical ocular administration as a part of a Phase 2 clinical trial.

### DETAILED DESCRIPTION

The description herein sets forth details to provide an understanding of various embodiments of the invention and is made with the understanding that the provided disclosures are an exemplification of the claimed subject matter without intending to limit the claims to specific embodiments. Accordingly, specific embodiments disclosed herein may be combined with other specific embodiments disclosed herein, including specific embodiments under various headings, which are provided for convenience and organization, but are not to be construed to limit the claims in any way.

All published documents cited herein are hereby incorporated by reference in their entirety.

As used herein, the singular forms “a”, “an” and “the” are intended to include the plural forms as well, unless the context clearly indicates otherwise.

As used herein, and unless otherwise specified, the term “about”, when used in connection with a numeric value or range of values which is provided to describe that the value or range of values may deviate to an extent deemed reasonable to one of ordinary skill in the art (e.g., a specific temperature or temperature range). For example, the term “about”, when used in this context, can, in some embodiments, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values. In some embodiments, the numeric value or range of values may vary by 5%.

Pterygium is an ocular surface disease, where a fibrovascular growth extends from the nasal or temporal conjunctiva across the limbus onto the cornea. The current understanding of the pathogenesis of pterygium is that multiple processes are involved and that these may include inherited factors, environmental triggers (UV light, viral infections), and factors that perpetuate its growth (cytokines, growth factors and matrix metalloproteinases). Among them, chronic UV exposure is typically understood to be the single most significant factor in the pathogenesis of pterygium. Pterygium affects about 10 million individuals in the US. Later-stage disease impairs vision and early to middle stage disease causes worry and anxiety about the disease (e.g., an ocular disease such as pterygium, hyperemia in pterygium, hyperemia, pinguecula, and/or pseudopterygium) and/or eye appearance (e.g., changed eye appearance as a result of the disease) in patients. The current standard of care is surgical removal of lesion tissue. However, rapidly growing lesions can recur in about 10% of patients after surgery.

Pterygium patients often experience symptoms and suffer from psychological stress and anxiety due to the disease and/or their altered eye appearances. Currently, there is no approved pharmacologic therapy to treat pterygia. Pterygium excision with conjunctival autograft transplantation is often the procedure of choice for definitive treatment of both primary and recurrent pterygia. While many of these lesions can be readily removed to the initial satisfaction of both surgeon and patient, the recurrence of pterygium could occur. Other than impaired vision, one of the main reasons patients elect surgical treatment is the feeling of unsightly appearance caused by the disease. In a recent study of 40 pterygia patients, Pandey analyzed the most common pterygium risk factors leading to the decision for surgical removal of pterygium (Pandey, 2017, semanticscholar.org/60f4/a88614fb8b12f2dd9a0b50dde324ae50adf3.pdf). One conclusion from the study was that for younger patients, the predominant reason for choosing surgical removal was that the patients are not content with the external appearance caused by pterygium. In a review article (Kaufman et al. 2013, sciencedirect.com/science/article/pii/S0039625702004630), the authors stated that “The presence of a pterygium is “disturbing to both the patient because of its unsightly appearance and the surgeon because of its tendency to recur.” Accordingly, the unsightly appearance induced bothersome or anxiety is an important factor for pterygia patients to seek surgery.

Pterygium is a multifactorial disease and several growth factors such as vascular endothelial growth factor (VEGF) and platelet-derived growth factor (PDGF) are potential pathological factors. However, no drug against these growth factors has been approved to treat the disease. Recently, the inventors of this disclosure have tested a small molecule multikinase inhibitor anti-angiogenesis and anti-fibrosis drug, nintedanib, in a Phase 2 clinical trial in pterygia patients. Nintedanib represents a class of multikinase inhibitors that confer anti-angiogenesis activity mainly by targeting VEGFR1 VEGFR2, VEGFR3, PDGFRalpha (PDGFRα) and PDGFRbeta (PDGFRβ), and/or FGFR1, FGFR2, FGFR3, and/or FGFR4.

One potential advantage of materials and methods disclosed herein can be to stop pterygium progression. Another potential advantage of materials and methods disclosed herein is to reduce the need for excision surgery, and possibly, lowering the risk of post-surgical disease recurrence. Yet another potential advantage of the materials and methods disclosed herein is the reduction of symptoms and/or signs of pterygium, hyperemia in pterygium, hyperemia, pinguecula, and/or pseudopterygium. One example of a symptom and/or sign is patients' worry or anxiety about the disease and/or appearance of their eye (e.g., changed eye appearance as a result of the disease). Another potential advantage of the materials and methods disclosed herein is improvement in the patients' quality of life.

In some embodiments, provided herein are methods of reducing worry or anxiety about an ocular disease and/or eye appearance (e.g., changed eye appearance as a result of the disease) of an affected eye in a subject, wherein the affected eye is affected with an ocular disease such as pterygium, hyperemia in pterygium, hyperemia, pinguecula, and/or pseudopterygium. The method can include identifying a subject with worry or anxiety about a disease (e.g., an ocular disease such as pterygium, hyperemia in pterygium, hyperemia, pinguecula, and/or pseudopterygium) and/or eye appearance (e.g., changed eye appearance associated with the disease) and administering a therapeutically effective amount of a multikinase inhibitor to the affected eye of the subject. In some embodiments, provided herein are methods of reducing worry or anxiety about an ocular disease and/or the appearance of an affected eye in a subject having the ocular disease. The method can include identifying a subject with worry or anxiety about the ocular disease and/or the appearance of the affected eye, and administering a therapeutically effective amount of a multikinase inhibitor to the affected eye of the subject for a period of time. In some embodiments, the ocular is disease selected from the group consisting of pterygium, hyperemia in pterygium, hyperemia, pinguecula, pseudopterygium, and a combination thereof. In some embodiments, a rating of worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease), as determined by a patient questionnaire, by the subject can decrease between two evaluation points. In some embodiments, a rating of worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease), as determined by a patient questionnaire, by the subject can decrease compared to a control. In some embodiments, a rating of worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease), as determined by a patient questionnaire, by the subject can decrease compared to a non-treated subject. In some embodiments, a rating of worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease), as determined by a patient questionnaire, by the subject can decrease compared to a baseline of the subject prior to administration of the multikinase inhibitor.

Also provided herein are methods of reducing one or more patient reported signs or symptoms in a patient having an eye affected with pterygium, hyperemia in pterygium, hyperemia, pinguecula, and/or pseudopterygium. The method can include administering a therapeutically effective amount of a multikinase inhibitor to the affected eye of the patient. Also provided herein are methods of reducing one or more patient reported signs or symptoms in a patient having an eye affected with an ocular disease, comprising administering a therapeutically effective amount of a multikinase inhibitor to the affected eye of the patient. In some embodiments, the ocular disease is selected from the group consisting of pterygium, hyperemia in pterygium, hyperemia, pinguecula, pseudopterygium or a combination thereof. In some embodiments, the patient reported sign or symptom is worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease) in the patient. In some embodiments, the one or more patient reported signs or symptoms can decrease between two evaluation points. In some embodiments, the one or more patient reported signs or symptoms can decrease compared to a control. In some embodiments, the one or more patient reported signs or symptoms can decrease compared to a non-treated subject. In some embodiments, the one or more patient reported signs or symptoms can decrease compared to a baseline of the subject prior to administration of the multikinase inhibitor.

Any of the evaluations of a sign or symptom (e.g., worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease)) can be reported as a rating on a numerical scale. In some cases, the reduction of a sign or a symptom can be at least about 10%, at least about 20%, at least about 30%, at least about 40%, at least about 50%, at least about 60%, or at least about 70% (e.g., as compared to a different evaluation point, a control, a non-treated patient, or a baseline of the patient prior to administration of the multikinase inhibitor). In some cases, the reduction of a sign or a symptom can be at least about 25%, at least about 30%, at least about 35%, at least about 40%, at least about 45%, at least about 50%, at least about 55%, at least about 60%, at least about 65%, at least about 70%, or at least about 75% (e.g., as compared to a different evaluation point, a control, a non-treated patient, or a baseline of the patient prior to administration of the multikinase inhibitor). A numerical scale can be any appropriate numerical scale. In some embodiments, the numerical scale can be a five-point scale (e.g., 0 to 4 or 1 to 5). In some embodiments, the numerical scale can be a ten-point scale (e.g., 0 to 9 or 1 to 10). In some embodiments, a numerical scale can be normalized to a five-point scale, e.g., for purposes of comparison to an evaluation using a five-point numerical scale. In some cases, the reduction of a sign or a symptom can be at least 0.3 points, 0.5 points, 0.7 points, 0.9 points, 1.0 points, 1.1 points, 1.3 points, or 1.5 points on the numerical scale (e.g., a five-point numerical scale, or normalized to a five-point numerical scale) (e.g., as compared to a different evaluation point, a control, a non-treated patient, or a baseline of the patient prior to administration of the multikinase inhibitor).

In some cases, any two appropriate evaluation points can be used. For example, the evaluation points (a) before administering a therapeutically effective amount of a multikinase inhibitor to an affected eye of the subject for a period of time and (b) after administering a therapeutically effective amount of a multikinase inhibitor to an affected eye of the subject to a period of time, can be used. As another example, the evaluation points (a) before administering one or more doses of a multikinase inhibitor to an affected eye of the subject for a period of time and (b) after administering one or more doses of a multikinase inhibitor to an affected eye of the subject, can be used. As yet another example, the evaluation points (a) before administering one or more doses of a multikinase inhibitor to an affected eye of the subject and (b) after administering one or more doses of a multikinase inhibitor to an affected eye of the subject, can be used.

In some cases, an evaluation of a sign or symptom (e.g., worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease)) can occur following administration of one or more doses of a multikinase inhibitor. One or more doses of a multikinase inhibitor can include any appropriate number of doses or dosing duration (which can also be called ‘a period of time’ for administration). In some cases, a period of time can be about 1 week, about 2 weeks, about 3 weeks, about 1 month, about 2 months, about 3 months, about 4 months, about 5 months, about 6 months, about 8 months, about 9 months, about 10 months, about 11 months, about 12 months (about 1 year), or longer. In some cases, a period of time can be about 3 months. In some cases, a period of time can be about 12 months.

Dosing can occur with any appropriate frequency. For example, dosing can occur once per day, twice per day, three times per day, four times per day, five times per day, or six times per day. In some embodiments, a multikinase inhibitor can be administered twice per day. In some embodiments, a multikinase inhibitor can be administered three times per day.

Any of the evaluations of a sign or symptom (e.g., worry or anxiety about a disease (e.g., an ocular disease such as pterygium, hyperemia in pterygium, hyperemia, pinguecula, and/or pseudopterygium) and/or eye appearance (e.g., changed eye appearance as a result of the disease)) can be based on a questionnaire (e.g., a patient questionnaire). The questionnaire can be any appropriate questionnaire. In some cases, a questionnaire can include a question regarding whether the worry about a disease (e.g., an ocular disease such as pterygium, hyperemia in pterygium, hyperemia, pinguecula, and/or pseudopterygium) and/or the appearance of the affected eye (e.g., changed eye appearance as a result of the disease) has impacted the quality of life of the subject in the last week. In some cases, the evaluation of a sign or symptom can be solely based on the question of whether the worry about a disease (e.g., an ocular disease such as pterygium, hyperemia in pterygium, hyperemia, pinguecula, and/or pseudopterygium) and/or the appearance of the affected eye has impacted the quality of life of the subject in the last week. In some cases, the questionnaire can include the category, “Has the appearance of the affected eye impacted the quality of life during the last week?”, and the question “Worry about eye appearance?”. For example, the question “Worry about eye appearance?” can be in the context of whether the subject's eye appearance has impacted the quality of life of the subject in the past week. Options can include: All of the time (e.g., associated with a numerical score of 5 on a 5-point scale from 1 to 5), most of the time (e.g., associated with a numerical score of 4 on a 5-point scale from 1 to 5), half of the time (e.g., associated with a numerical score of 3 on a 5-point scale from 1 to 5), some of the time (e.g., associated with a numerical score of 2 on a 5-point scale from 1 to 5), none of the time (e.g., associated with a numerical score of 1 on a 5-point scale from 1 to 5), or not applicable (e.g., “NA”). In some cases, the questionnaire can include the category, “Has the appearance of the affected eye impacted the quality of life during the last week?”, and the question “Worry about eye appearance?”, and the evaluation of a sign or symptom (e.g., worry or anxiety about eye appearance) can be solely based on this question. In some embodiments, questionnaire can be derived, at least in part, from the Visual Functioning Questionnaire-25 (VFQ-25) questionnaire (National Eye Institute, 2000). In some embodiments, the questionnaire can be derived, at least in part, from the Ocular Surface Disease Index (OSDI) questionnaire (Schiffman R M, Christianson M D, Jacobsen G, Hirsch J D, Reis B L. Reliability and validity of the Ocular Surface Disease Index. Arch Ophthalmol. 2000; 118 (5): 615-621. doi: 10.1001/archopht.118.5.615).

Any appropriate multikinase inhibitor can be used. In some cases, the multikinase inhibitor is selected from afatinib, amuvatinib, axitinib, cabozantinib, canertinib, cediranib, ceritinib, crenolanib, crizotinib, dabrafenib, dacomitinib, dasatinib, erlotinib, foretinib, gefitinib, golvatinib, ibrutinib, icotinib, idelalisib, imatinib, lapatinib, lenvatinib, neratinib, nilotinib, nintedanib, palbociclib, pazopanib, ponatinib, quizartinib, regorafenib, ruxolitinib, sorafenib, sunitinib, tandutinib, tivantinib, tivozanib, trametinib, vandetanib, vatalanib, vemurafenib, or combinations thereof. In some cases, the multikinase inhibitor is selected from axitinib, nintedanib, regorafenib, and pazopanib. In some cases, the multikinase inhibitor is axitinib. In some cases, the multikinase inhibitor is nintedanib. In some cases, the multikinase inhibitor is regorafenib. In some case, the multikinase inhibitor is pazopanib. In some cases, multikinase inhibitor is a free base. In some cases, the multikinase inhibitor is a pharmaceutically acceptable salt.

The multikinase inhibitor (e.g., nintedanib or axitinib or pazopanib) can be administered in any appropriate concentration. In some cases, the multikinase inhibitor can be administered in an amount from about 0.001% to about 10.0% (w/w). In some cases, the multikinase inhibitor can be administered in an amount of from about 0.005% to about 2% (w/w), from about 0.001% to about 1% (w/w), from about 0.001% to about 0.005% (w/w), from about 0.005% to about 0.01% (w/w), from about 0.01% to about 0.05% (w/w), from about 0.05% to about 0.1% (w/w), from about 0.01% to about 1% (w/w), from about 0.05% to about 0.5%, from about 0.01% to about 0.8% (w/w), from about 0.3% to about 0.7% (w/w), from about 0.4% to about 0.6% (w/w), from about 0.1% to about 10% (w/w), from about 0.1% to about 0.5% (w/w), from about 0.2% to about 8% (w/w), from about 0.4% to about 5% (w/w), or from about 0.4% to about 2% (w/w).

The present disclosure includes data from a Phase 2, randomized clinical trial.

Certain embodiments of this invention are described herein. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The present invention is not limited to that precisely as shown and described. Specific embodiments disclosed herein may be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein.

### REFERENCES

Kaufman S C, Jacobs D S, Lee W B, Deng S X, Rosenblatt M I, Shtein R M. Options and adjuvants in surgery for pterygium: a report by the American Academy of Ophthalmology. Ophthalmology. 2013 January; 120(1):201-8.
Pandey A N. Assessment of the Most Common Pterygium Symptoms and Risk Factors Leading to the Decision for its Surgical Removal-A long term study. EC Ophthalmology. 2017; 5.6 (2017): 231-234

### Examples

### Example 1

A Phase 2a Multicenter, Randomized, Vehicle-Controlled, Dose Escalating Study to Evaluate the Safety, Efficacy and Pharmacokinetics of Nintedanib Ophthalmic Solution in Patients with Primary or Recurrent Pterygium

Objectives of the study were to evaluate ocular and systemic safety of nintedanib and its efficacy in reducing pterygium vascularity in primary or recurrent patients. Several other secondary endpoints were also assessed, including the assessment of symptoms and life impacts by using a questionnaire based on a numerical 1-5 scale. The double-masked, vehicle-controlled, parallel study was conducted with 28 days TID repeat ocular dosing of vehicle and 0.2% nintedanib.

Results

The disclosure focused on the relevant questionnaire endpoint that included 15 questions regarding the ocular symptoms, vision related functioning and the impact on life quality in patients. The questionnaire was derived from the validated Visual Functioning Questionnaire-25 (VFQ-25) (National Eye Institute, 2000) and the validated Ocular Surface Disease Index (OSDI) questionnaire (Schiffman R M, Christianson M D, Jacobsen G, Hirsch J D, Reis B L. Reliability and validity of the Ocular Surface Disease Index. Arch Ophthalmol. 2000; 118 (5): 615-621.). The patients were asked to rate severity using a 5-point scale, with 5 being the most severe.

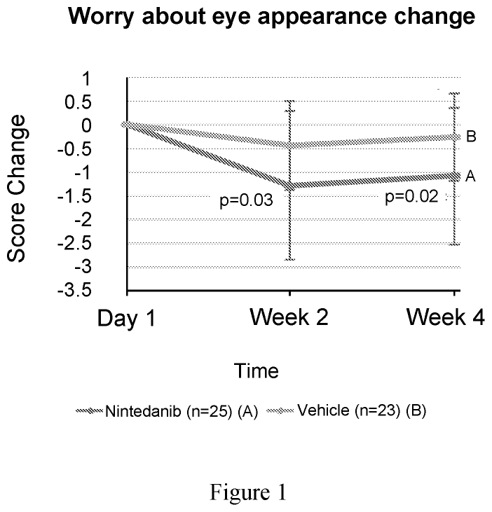

The analysis of the questionnaire data revealed surprising results. For the question “Worry about eye appearance?” under the category, “Has the appearance of the affected eye impacted the quality of life during the last week?”, it was observed that the drug group showed significantly better improvements than the vehicle group on the frequency of worry about eye appearance as shown in FIG. 1. Questionnaire surveys on symptoms and psychological assessments are often variable. It is surprising that a question or set of questions could discern a difference in worrying or anxiety about appearance between drug and vehicle. The fact that only one out of fifteen questions showed a significant difference also concur with this unexpected result. As discussed previously, pterygia patients' concern about their eye appearance has a major role in surgical decisions. The improvement of this concern with a multikinase inhibitor such as nintedanib will address a clear unmet medical need in pterygia treatment.

1. A method of reducing worry or anxiety about an ocular disease and/or the appearance of an affected eye in a subject having the ocular disease, the method comprising:
identifying a subject with worry or anxiety about the ocular disease and/or the appearance of the affected eye; and
administering a therapeutically effective amount of a multikinase inhibitor to the affected eye of the subject for a period of time,
wherein the affected eye is affected with the ocular disease selected from the group consisting of pterygium, hyperemia in pterygium, hyperemia, pinguecula, pseudopterygium, and a combination thereof.

2. The method of claim 1, wherein a rating of worry or anxiety about the ocular disease and/or the appearance of the affected eye, as determined by a patient questionnaire, by the subject decreases between (a) before administering a therapeutically effective amount of a multikinase inhibitor to the affected eye of the subject for a period of time and (b) after administering a therapeutically effective amount of a multikinase inhibitor to the affected eye of the subject for a period of time.

3. The method of claim 2, wherein the rating is on a numerical scale.

4. The method of claim 3, wherein the rating decreases by at least about 25%.

5.-14. (canceled)

15. The method of claim 3, wherein the numerical scale is a five-point scale.

16. The method of claim 3, wherein the rating decreases by at least about 0.5.

17.-21. (canceled)

22. The method of claim 2, wherein the rating of worry or anxiety about the ocular disease and/or the appearance of the affected eye, as determined by a patient questionnaire, is based on a question regarding whether the worry about the ocular disease and/or the appearance of the affected eye has impacted the quality of life of the subject in the last week.

23. The method of claim 2, wherein the rating of worry or anxiety about the ocular disease and/or the appearance of the affected eye, as determined by a patient questionnaire, is solely based on a question regarding whether the worry about the ocular disease and/or the appearance of the affected eye has impacted the quality of life of the subject in the last week.

24. The method of claim 1, wherein the multikinase inhibitor is selected from the group consisting of afatinib, amuvatinib, axitinib, cabozantinib, canertinib, cediranib, ceritinib, crenolanib, crizotinib, dabrafenib, dacomitinib, dasatinib, erlotinib, foretinib, gefitinib, golvatinib, ibrutinib, icotinib, idelalisib, imatinib, lapatinib, lenvatinib, neratinib, nilotinib, nintedanib, palbociclib, pazopanib, ponatinib, quizartinib, regorafenib, ruxolitinib, sorafenib, sunitinib, tandutinib, tivantinib, tivozanib, trametinib, vandetanib, vatalanib, vemurafenib, and a combination thereof.

25.-29. (canceled)

30. The method of claim 1, wherein the multikinase inhibitor is a free base.

31. The method of claim 1, wherein the multikinase inhibitor is a pharmaceutically acceptable salt.

32. A method of reducing one or more patient reported signs or symptoms in a patient having an eye affected with an ocular disease, comprising administering a therapeutically effective amount of a multikinase inhibitor to the affected eye of the patient, wherein the ocular disease is selected from the group consisting of pterygium, hyperemia in pterygium, hyperemia, pinguecula, pseudopterygium and a combination thereof.

33. The method of claim 32, wherein the patient reported sign or symptom is worry or anxiety about the ocular disease and/or the appearance of the affected eye in the patient.

34. The method of claim 32, wherein reducing the one or more patient reported signs or symptoms comprises a measured reduction in the one or more patient reported signs or symptoms of at least about 10%, at least about 20%, at least about 30%, at least about 40%, at least about 50%, at least about 60%, or at least about 70% as compared to a control, a non-treated patient, or a baseline of the patient prior to administration of the multikinase inhibitor.

35.-38. (canceled)

39. The method of claim 32, wherein reducing the one or more patient reported signs or symptoms comprises a measured reduction in the one or more patient reported signs or symptoms of at least about 25%.

40.-49. (canceled)

50. The method of claim 32, wherein reducing the one or more patient reported signs or symptoms comprises a measured reduction in the one or more patient reported signs or symptoms on a five-point numerical scale.

51. The method of claim 32, wherein reducing the one or more patient reported signs or symptoms comprises a measured reduction of least 0.5 in the one or more patient reported signs or symptoms on a numerical scale.

52.-56. (canceled)

57. The method of claim 32, wherein the patient reported sign or symptom is determined by a patient questionnaire that includes a question regarding whether worry about the ocular disease and/or the appearance of the affected eye has impacted the quality of life of the subject in the last week.

58. The method of claim 32, wherein the patient reported sign or symptom is determined by a patient questionnaire and is solely based on a question regarding whether worry about the ocular disease and/or the appearance of the affected eye has impacted the quality of life of the subject in the last week.

59. The method of claim 32, wherein the multikinase inhibitor is selected from the group consisting of afatinib, amuvatinib, axitinib, cabozantinib, canertinib, cediranib, ceritinib, crenolanib, crizotinib, dabrafenib, dacomitinib, dasatinib, erlotinib, foretinib, gefitinib, golvatinib, ibrutinib, icotinib, idelalisib, imatinib, lapatinib, lenvatinib, neratinib, nilotinib, nintedanib, palbociclib, pazopanib, ponatinib, quizartinib, regorafenib, ruxolitinib, sorafenib, sunitinib, tandutinib, tivantinib, tivozanib, trametinib, vandetanib, vatalanib, vemurafenib, and a combination thereof.

60.-66. (canceled)
